(19) United States
(12) Patent Application Publication
(10) Pub. No.: US 2026/0290616 A1
(43) Pub. Date: Sep. 24, 2026

(54) SYSTEMS AND METHODS FOR ADVANCED PALLIATIVE CARE INTEGRATED WITH ELECTRONIC HEALTH RECORDS

(71) Applicants: Washington University, St. Louis, MO (US); BJC Healthcare, St. Louis, MO (US)

(72) Inventors: Nathan Moore, St. Louis, MO (US); Patrick White, St. Louis, MO (US); Randi Foraker, St. Louis, MO (US)

(21) Appl. No.: 19/689,490

(22) Filed: May 27, 2026

Related U.S. Application Data
(63) Continuation of application No. 17/934,634, filed on Sep. 23, 2022.
(60) Provisional application No. 63/247,510, filed on Sep. 23, 2021.

(51) Int. Cl. G16H 50/30; G16H 10/60

(52) U.S. Cl. CPC G16H 50/30; G16H 10/60

(57) ABSTRACT

A system is provided. The system includes a patient analysis (“PA”) computer device including at least one processor in communication with at least one memory device. The at least one processor is programmed to a) store a patient analysis model, b) receive a patent identifier associated with the patient; c) retrieve a plurality of claim information associated with the patient identifier; d) retrieve a plurality of electronic healthcare records (EHR) associated with the patient identifier; e) execute the patient analysis model to determine a patient risk score based on the plurality of claim information and the plurality of EHR; and f) present information about the patient to a healthcare provider.

### CROSS-REFERENCE TO RELATED APPLICATIONS

This application is a continuation in part of U.S. patent application Ser. No. 17/934,634, filed on Sep. 23, 2022, which claims priority to U.S. Provisional Patent Application Ser. No. 63/247,510, filed Sep. 23, 2021. U.S. patent application Ser. No. 17/934,634 and U.S. Provisional Patent Application Ser. No. 63/247,510 are hereby incorporated by reference in their entirety.

### BACKGROUND

The field of the present disclosure relates generally to advanced palliative care integrated with electronic health records and, more specifically, to training and managing artificial intelligence systems to manage palliative care resources based on electronic health records.

Although many seriously ill patients wish to avoid aggressive and burdensome care, many receive invasive procedures and therapies in the final months of their lives with limited benefit. Such aggressive care near the end of life is associated with reduced quality of life for patients, excessive end-of-life medical spending, and a high proportion of patients dying in the hospital or other medical facility rather than at home.

Palliative care is a holistic approach to care administered at any point in a serious illness that offers patients support with intensive symptom management and complex medical decision-making. However, in many cases, these patients typically desire less aggressive care. Palliative care is associated with improved patient quality of life, reduced hospital readmissions, decreased hospital length of stay, and reduced total cost of care. Despite multiple studies demonstrating that the majority of patients in the last of year of life experience a high and escalating symptom burden and considerable need for support with advanced care planning, providers and health care systems have struggled to proactively identify and support these high-risk patients and their families.

Improving the use of palliative care has been identified as a key driver of success for value-based reimbursement models such as accountable care organizations (ACOs), Medicare Advantage plans, and capitated insurance plans. One study of managed care patients provided with home palliative care support found a mean savings of $3908 per member per month in the last three months of life compared with propensity-matched controls, with hospice participation increasing from 25% to 70% and median hospice length of stay increasing from 9 to 34 days. Currently, the majority of patients who would benefit from palliative care services are either referred late in their disease course or not at all, so there has been intense interest in developing and implementing prognostic tools to proactively identify patients near the end of life who may benefit from palliative care services.

The public health crisis caused by the COVID-19 pandemic has further altered healthcare delivery to both COVID and non-COVID patients. The COVID-19 pandemic has placed significant strain on the American healthcare system. Unprecedented shortages of medical resources and systemic measures to minimize exposure risk have impacted healthcare delivery not only to COVID patients, but also to patients without COVID-19 who have faced treatment delays and changes in standards of care. Identifying patients at high risk for mortality may assist medical decision-making in the face of such clinical uncertainty. Accurate prognostication in the era of COVID, however, remains a significant clinical challenge. Many of the previously published mortality scores are restricted to specific patient populations or diseases, whose standard treatments may have been altered by COVID-19. In addition, the changing mortality rates associated with COVID-19 have made even subjective prognostication unreliable.

An accurate prognostic model that can be widely applied to hospitalized patients with and without COVID-19 infection would be beneficial for improved inpatient care.

### BRIEF DESCRIPTION

In one aspect, a system is provided. The system includes patient analysis (“PA”) computer device including at least one processor in communication with at least one memory device. The at least one processor is programmed to store a patient analysis model. The at least one processor is also programmed to receive a patent identifier associated with the patient. The at least one processor is further programmed to retrieve a plurality of claim information associated with the patient identifier. In addition, the at least one processor is programmed to retrieve a plurality of electronic healthcare records (EHR) associated with the patient identifier. Moreover, the at least one processor is programmed to execute the patient analysis model to determine a patient risk score based on the plurality of claim information and the plurality of EHR. Furthermore, the at least one processor is programmed to present information about the patient to a healthcare provider. The system may include additional, less, or alternate functionality, including that discussed elsewhere herein.

In another aspect, a method is provided. The method is implemented with a patient analysis (“PA”) computer device comprising at least one processor in communication with at least one memory device. The method includes storing a patient analysis model. The method also includes receiving a patent identifier associated with the patient. The method further includes retrieving a plurality of claim information associated with the patient identifier. In addition, the method includes retrieving a plurality of electronic health records (EHR) associated with the patient identifier. Moreover, the method includes executing the patient analysis model to determine a patient risk score based on the plurality of claim information and the plurality of EHR. Furthermore, the method includes present information about the patient to a healthcare provider. The method may include additional, less, or alternate functionality, including that discussed elsewhere herein.

In a further aspect, a computer device is provided. The computer device includes at least one processor in communication with at least one memory device. The at least one processor is programmed to store a patient analysis model. The at least one processor is also programmed to receive a patent identifier associated with the patient. The at least one processor is further programmed to retrieve a plurality of claim information associated with the patient identifier. In addition, the at least one processor is programmed to retrieve a plurality of electronic health records (EHR) associated with the patient identifier. Moreover, the at least one processor is programmed to execute the patient analysis model to determine a patient risk score based on the plurality of claim information and the plurality of EHR. Furthermore, the at least one processor is programmed to present information about the patient to a healthcare provider. The computer device may include additional, less, or alternate functionality, including that discussed elsewhere herein.

### BRIEF DESCRIPTION OF THE DRAWINGS

The Figures described below depict various aspects of the systems and methods disclosed therein. It should be understood that each Figure depicts an embodiment of a particular aspect of the disclosed systems and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawing's arrangements, which are presently discussed, it being understood, however, that the present embodiments are not limited to the precise arrangements and instrumentalities shown, wherein:

FIG. 1 illustrates a block diagram of a structure for a deep learning model, in accordance with one embodiment of the present disclosure.

FIG. 2A illustrates a graph of a receiver operating characteristic (ROC) curve for predicting performance of the deep learning model shown in FIG. 1.

FIG. 2B illustrates a graph of a precision recall curve for predicting performance of the deep learning model shown in FIG. 1.

FIGS. 3A-3F illustrate a plurality of graphs of performance evaluation of the deep learning model shown in FIG. 1 based on different subgroups of patients.

FIG. 4 illustrates a process for analyzing communication interfaces using the system shown in FIG. 5.

FIG. 5 depicts a simplified block diagram of an exemplary computer system for implementing the processes shown in FIG. 4.

FIG. 6 depicts an exemplary configuration of client computer devices, in accordance with one embodiment of the present disclosure.

FIG. 7 illustrates an example configuration of the server system, in accordance with one embodiment of the present disclosure.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

The implementations described herein relate to advanced palliative care systems and, more specifically, to training and managing artificial intelligence systems to manage palliative care of patients. More specifically, a patient analysis (“PA”) computer device is provided for training and controlling advanced palliative care systems.

The systems and methods in this disclosure describe a deep learning model can effectively predict short-term mortality or hospice outcomes on the second day of hospital admission for patients with and without COVID-19. Early identification of high-risk inpatients can be used to assist clinical decision-making.

The systems and methods described herein illustrate a deep learning model on trained on and implemented on electronic health records (EHRs) and administrative claims for a large number of patients. The use of administrative claims accounts for cases there the patients has receive care in multiple medical systems, as most patients do. The claims data provide a richer picture for each patient, although the information can lag by 3 to 6 months. While this information may not be available in real-time, it may be used in some embodiments, for training purposes. In some further embodiments, the model is trained with just the EHR data.

A deep learning method and model can then be used to predict mortality of inpatients and outpatients in addition to identifying patients to be considered for palliative care. One use of this system would be to provide a clinical referral system for daily use to assist physicians in identifying patients who could benefit from palliative care and/or hospice care. This referral system could provide information about the risk level for each incoming patient to allow healthcare providers to determine the appropriate level of care suggested for each patient.

One potential solution to the challenge of dealing with analysis is through machine learning. By leveraging data on a scale orders of magnitude higher than traditional models, machine learning techniques can be used to efficiently and accurately predict outcomes in a variety of healthcare settings. To that end, the systems and methods described herein use a deep learning algorithm using large-volume EHR data and administrative claims to predict patient mortality. This model can be further adapted to the COVID-19 crisis by replacing administrative claims with vital signs, medications, and laboratory data from the patient's first hospital day. By utilizing both historical diagnoses and procedures as well as acute physiologic derangements and ongoing treatments in a single algorithm, this system can accurately predict mortality in patients with and without COVID-19 for general use across an entire hospital system. Since the claims data can lag three to six months, the model can use the claims data for a historical perspective on the patient and use the vital signs, medications, and laboratory data from the patient's first hospital day for current data.

The machine learning techniques can include, but are not limited to, long short-term memory (LSTM), deep neural networks (DNN), random forest (RF), and logistic regression (LR).

In the exemplary embodiment, patient data can be collected for historical data from admissions from academic and community-based hospitals. In the exemplary embodiment, hospitalizations longer than 24 hours can be included for feature extraction. Admissions to psychiatry, labor/delivery, and bone marrow transplant units can be excluded. For patients with multiple admissions in the same data set, one admission can be selected (randomly or otherwise) for feature extraction to reduce selection bias. For the purposes of this analysis, a patient can be considered for the outcome of interest if the patient died while in the hospital, was discharged to hospice, or had a recorded date of death within 30 days of discharge. In one set of historical analysis data, 46,206 admissions with hospitalizations longer than 24 hours and identifiable mortality outcomes within 30 days post-discharge were identified. A total of 35,521 unique patients were included in the analysis.

Subgroup analysis can be performed in multiple patient subgroups to evaluate for model accuracy and bias. In one example, these subgroups can be defined by, but are not limited to, COVID status (COVID+ and COVID−), ICU admission within the first 24 hours of admission (ICU+ and ICU−), and other information, such as demographics.

In at least one embodiment, encounter records for each patient available up to 24 hours after time of admission are extracted from the EHR data. Features can include, but are not limited to, problem lists, demographics, social history, diagnosis codes, procedure codes, medication codes, inpatient medication lists, lab results, vital signs, and substance abuse history. In these embodiments, all features are sorted in a time increasing order.

In the exemplary embodiments, diagnosis, procedure, and medication codes are mapped to a 32-dimensional vector space, such as, by the technique of Word2Vec. For example, a Python Genism Word2Vec model can be used with the following hyperparameters: size (embedding dimension) of 32, window (the maximum distance between a target word and all words around it) of 5, min_count (the minimum number of words counted when training the model) of 1, and the sg (training algorithm) was CBOW (the continuous bag of words). Each feature can then be represented by a 32-dimensional numerical vector. For labs and vital signs, the testing names and related numerical values can be added as features. In some embodiments, age and gender can also be included as features in the model. Each individual patient can be assigned a distinct numerical vector representing their combination of codes and numerical variables.

In the exemplary embodiment, when training the model, patients are randomly divided into training (80%), validation (10%), and testing (10%) data sets. In the above example, a total of 28,417 patients would be in the training data set and 3,552 patients would each be in the validation and testing data sets. Model training can be followed by model validation on the corresponding data sets. Model performance can be analyzed based on the validation data set to select the final model to be tested in the testing data set.

In some embodiments, the system can limit the number of events analyzed for each patient. For example, the events could be limited to the patient's most recent 500 events. Furthermore, for training, null events could be added for patients with less than 500 events. In other embodiments, all events of a patient may be analyzed. In still further embodiments, events may be weighted differently based on the amount of time since the event's occurrence.

Described herein are computer systems such as the PA computer devices and related computer systems. As described herein, all such computer systems include a processor and a memory. However, any processor in a computer device referred to herein may also refer to one or more processors wherein the processor may be in one computing device or a plurality of computing devices acting in parallel. Additionally, any memory in a computer device referred to herein may also refer to one or more memories wherein the memories may be in one computing device or a plurality of computing devices acting in parallel.

As used herein, a processor may include any programmable system including systems using micro-controllers; reduced instruction set circuits (RISC), application-specific integrated circuits (ASICs), logic circuits, and any other circuit or processor capable of executing the functions described herein. The above examples are example only and are thus not intended to limit in any way the definition and/or meaning of the term “processor.”

As used herein, the term “database” may refer to either a body of data, a relational database management system (RDBMS), or to both. As used herein, a database may include any collection of data including hierarchical databases, relational databases, flat file databases, object-relational databases, object-oriented databases, and any other structured collection of records or data that is stored in a computer system. The above examples are example only, and thus are not intended to limit in any way the definition and/or meaning of the term database. Examples of RDBMS' include, but are not limited to including, Oracle® Database, MySQL, IBM® DB2, Microsoft® SQL Server, Sybase®, and PostgreSQL. However, any database may be used that enables the systems and methods described herein. (Oracle is a registered trademark of Oracle Corporation, Redwood Shores, California; IBM is a registered trademark of International Business Machines Corporation, Armonk, New York; Microsoft is a registered trademark of Microsoft Corporation, Redmond, Washington; and Sybase is a registered trademark of Sybase, Dublin, California.)

In another embodiment, a computer program is provided, and the program is embodied on a computer-readable medium. In an example embodiment, the system is executed on a single computer system, without requiring a connection to a server computer. In a further example embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Cisco Systems, Inc. located in San Jose, CA). In yet a further embodiment, the system is run on a Mac OS® environment (Mac OS is a registered trademark of Apple Inc. located in Cupertino, CA). In still yet a further embodiment, the system is run on Android® OS (Android is a registered trademark of Google, Inc. of Mountain View, CA). In another embodiment, the system is run on Linux® OS (Linux is a registered trademark of Linus Torvalds of Boston, MA). The application is flexible and designed to run in various different environments without compromising any major functionality.

The application is flexible and designed to run in various different environments without compromising any major functionality. In some embodiments, the system includes multiple components distributed among a plurality of computer devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes. The present embodiments may enhance the functionality and functioning of computers and/or computer systems.

As used herein, an element or step recited in the singular and proceeded with the word “a” or “an” should be understood as not excluding plural elements or steps, unless such exclusion is explicitly recited. Furthermore, references to “example embodiment” or “one embodiment” of the present disclosure are not intended to be interpreted as excluding the existence of additional embodiments that also incorporate the recited features.

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are example only and are thus not limiting as to the types of memory usable for storage of a computer program.

Furthermore, as used herein, the term “real-time” refers to at least one of the time of occurrence of the associated events, the time of measurement and collection of predetermined data, the time to process the data, and the time of a system response to the events and the environment. In the embodiments described herein, these activities and events occur substantially instantaneously.

The systems and processes are not limited to the specific embodiments described herein. In addition, components of each system and each process can be practiced independent and separate from other components and processes described herein. Each component and process also can be used in combination with other assembly packages and processes.

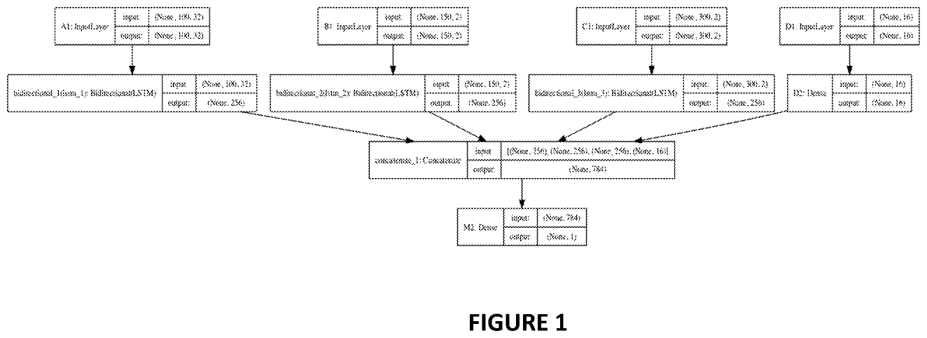

FIG. 1 illustrates a block diagram of a structure for a deep learning model, in accordance with one embodiment of the present disclosure.

The aforementioned numerical vectors were the inputs of the deep learning model. These inputs were structured into four groups: (1) embedding vectors of diagnosis codes, procedure codes, and medication codes for each patient with a dimension of (100, 32), where 100 denoted the most recent 100 codes and 32 was the dimension of embedding vectors; (2) numerical variables from lab results for each patient with a dimension of (150, 2), where 150 was the number of most recent lab records, and 2 was the lab test names and related values; (3) numerical variables from vital signs results for each patient with a dimension of (300, 2), where 300 was the number of most recent vital sign records, and 2 was the vital sign names and related values; and (4) demographic and social history variables for each patient with a dimension of 16, which represented 16 variables including, but not limited to, age, sex, admission location, COVID−19 infection flag, ICU flag, and social history lists.

The deep-learning model is composed of a combination of four models representing the above groups. Three models are bidirectional long short-term memory (LSTM) models and the final is a neural network model (FIG. 1). A binary cross-entropy loss function can be employed as the output layer and a Sigmoid function was used as the activation function for the hidden layer. An Adam optimizer can used to optimize the model, such as with a mini-batch size of 256 samples.

Deep learning model performance can be evaluated using a variety of metrics, including area under the receiver operator characteristic (AUROC) curves, precision-recall curves, overall accuracy, sensitivity, specificity, precision, F1-score, and negative predicted value (NPV). 95% confidence intervals (95% CI) can also calculated for each metric. The model can also be evaluated under a series of discrimination thresholds or cut-offs, ranging from 0.1 to 0.9 in increments of 0.1. Model performance in the three previously defined patient sub-groups can also evaluated using the same metrics.

TABLE 1

| | No Mortality/ | Mortality/ | |
|---|---|---|---|
| | Hospice | Hospice | |
| Characteristic | Outcome | Outcome | Total |
| Number of patients n | 32682 (92.0) | 2839 (8.0) | 35521 |
| Age mean (std) | 61.2 (17.5) | 72.6 (15.0) | 62.1 (17.6) |
| Sex n (%) | | | |
| Male | 16191 (49.5) | 1451 (51.1) | 17642 (49.7) |
| Female | 16490 (50.5) | 1388 (48.9) | 17878 (50.3) |
| Unknown | 1 (0.0) | 0 (0.0) | 1 (0.0) |
| Race n (%) | | | |
| White | 23616 (72.3) | 2074 (73.1) | 25690 (72.3) |
| Black | 8438 (25.8) | 669 (23.6) | 9107 (25.6) |
| Asian | 257 (0.8) | 22 (0.8) | 279 (0.8) |
| Other | 371 (1.1) | 74 (2.6) | 445 (1.0) |
| COVID flag n (%) | | | |
| COVID (+) | 1587 (4.9) | 433 (15.3) | 2020 (5.7) |
| COVID (−) | 31095 (95.1) | 2406 (84.7) | 33501 (94.3) |
| ICU flag n (%) | | | |
| ICU (+) | 4475 (13.7) | 1089 (38.4) | 5564 (15.7) |
| ICU (−) | 28207 (86.3) | 1750 (61.6) | 29957 (84.3) |
| Lab Values, most | | | |
| prevalent, mean (std) | | | |
| Hematocrit | 36.0 (7.3) | 35.0 (7.5) | 35.5 (7.2) |
| Hemoglobin | 11.7 (2.4) | 11.9 (2.6) | 11.7 (2.5) |
| Sodium | 138.0 (6.9) | 137.6 (5.6) | 137.6 (5.5) |
| Glucose | 153.6 (107.8) | 135.5 (82.0) | 147.9 (88.6) |
| Creatinine | 1.6 (1.8) | 1.8 (2.2) | 1.5 (1.8) |
| Creatinine clearance | 49.4 (25.6) | 48.2 (25.3) | 54.6 (28.3) |
| Chloride | 101.6 (7.3) | 101.5 (6.8) | 101.9 (6.5) |
| Urea Nitrogen (BUN) | 25.3 (22.9) | 26.6 (20.0) | 23.6 (20.3) |
| Calcium | 9.0 (0.7) | 8.9 (0.7) | 8.9 (0.8) |
| Carbon dioxide | 24.0 (4.9) | 23.7 (5.2) | 23.8 (4.3) |

Table 1 illustrates the characteristics of an example study population. In this example study population, approximately 8% of patients were positive for the composite outcome of in-hospital mortality, 30-day mortality, or hospice discharge, while 92% of patients were negative for the outcome of interest. Patients who met the composite outcome had an average age of 72.6 years compared to 61.2 years in the negative group. Sex and race distributions were similar between groups. Patients with COVID−19 infection were more likely to experience the mortality/hospice outcome than patients without COVID (21.4% vs 5.2%); as a result, while only 5.7% of patients in the study population had COVID−19 infection, this subgroup comprised 15.3% of the patients who died or were discharged to hospice. 38.4% of patients who met the mortality/hospice outcome were admitted to an ICU within the first 24 hours of admission.

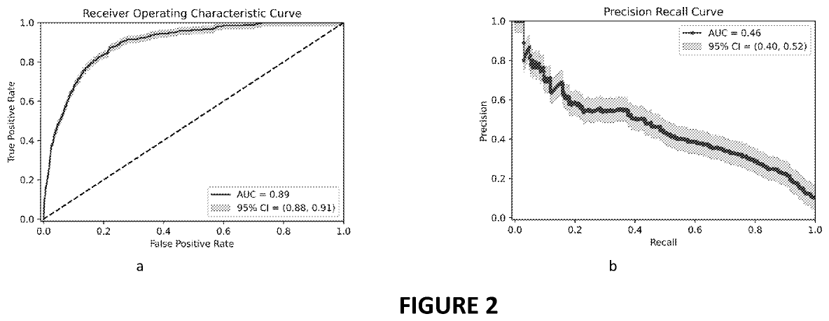

FIG. 2A illustrates a graph of a receiver operating characteristic (ROC) curve for predicting performance of the deep learning model shown in FIG. 1. FIG. 2B illustrates a graph of a precision recall curve for predicting performance of the deep learning model shown in FIG. 1.

TABLE 2

| Cut-offs | Accuracy | Sensitivity | Specificity | Precision | F1-score | NPV |
|---|---|---|---|---|---|---|
| 0.1 | 0.86 | 0.76 | 0.86 | 0.32 | 0.45 | 0.98 |
| 0.2 | 0.91 | 0.52 | 0.94 | 0.42 | 0.46 | 0.96 |
| 0.25 | 0.92 | 0.44 | 0.96 | 0.48 | 0.46 | 0.95 |
| 0.3 | 0.93 | 0.38 | 0.97 | 0.54 | 0.44 | 0.95 |
| 0.4 | 0.93 | 0.23 | 0.98 | 0.55 | 0.32 | 0.94 |
| 0.5 | 0.93 | 0.13 | 0.99 | 0.64 | 0.21 | 0.93 |
| 0.6 | 0.93 | 0.1 | 1.0 | 0.76 | 0.17 | 0.93 |
| 0.7 | 0.93 | 0.05 | 1.0 | 0.81 | 0.09 | 0.93 |
| 0.8 | 0.93 | 0.02 | 1.0 | 1.0 | 0.04 | 0.93 |
| 0.9 | 0.0 | 0.0 | 0.0 | 0.0 | 0.0 | 0.0 |

Table 2 shows the deep learning model performance at different thresholds or cut-offs of predicted probabilities. The model had the highest sensitivity (76%) with lower precision (32%) and specificity (86%) when using the lowest cut-off of 0.1. With increasing cut-off values, the sensitivity and NPV of the model generally decreased with a corresponding increase in specificity and precision.

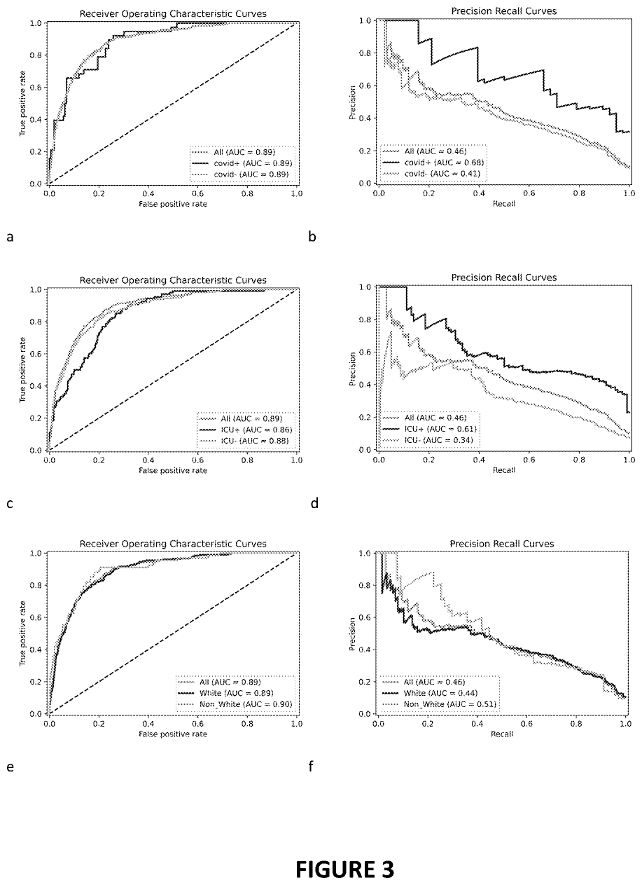

FIGS. 3A-3F illustrate a plurality of graphs of performance evaluation of the deep learning model shown in FIG. 1 based on different subgroups of patients. FIGS. 3A-3F show the model's prediction performance for different subgroups by AUROC and precision recall. The AUROC in the COVID+subgroup was 0.89 (0.83-0.94), compared to an AUROC of 0.89 (0.87-0.91) in the COVID-subgroup. The AUC for the precision recall curve was 0.68 (0.52, 0.81) for the COVID+subgroup and 0.41 (0.35, 0.48) for the COVID-subgroup.

Model performance evaluation for different subgroups of patients by AUROC (3A, 3C, 3Ee) and precision recall (3B, 3D, 3F). The first row (3A, 3B) is for COVID+ and COVID−; the second row (3C, 3D) is for ICU+ and ICU−; and the third row (3E, 3F) is for White and Non-White.

TABLE 3

| Subgroups | Cut-offs | Accuracy | Sensitivity | Specificity | Precision | f1-score | NPV |
|---|---|---|---|---|---|---|---|
| COVID+ | 0.1 | 0.77 | 0.92 | 0.73 | 0.45 | 0.6 | 0.97 |
| | 0.2 | 0.82 | 0.71 | 0.84 | 0.52 | 0.6 | 0.92 |
| | 0.3 | 0.86 | 0.66 | 0.91 | 0.62 | 0.64 | 0.92 |
| | 0.4 | 0.84 | 0.39 | 0.94 | 0.62 | 0.48 | 0.87 |
| | 0.5 | 0.85 | 0.29 | 0.98 | 0.79 | 0.42 | 0.85 |
| | 0.6 | 0.84 | 0.21 | 0.99 | 0.8 | 0.33 | 0.84 |
| | 0.7 | 0.83 | 0.16 | 0.99 | 0.86 | 0.27 | 0.83 |
| | 0.8 | 0.82 | 0.05 | 1.0 | 1.0 | 0.1 | 0.82 |
| | 0.9 | 0.81 | 0.0 | 1.0 | 0.0 | 0.0 | 0.81 |
| COVID− | 0.1 | 0.86 | 0.73 | 0.87 | 0.3 | 0.42 | 0.98 |
| | 0.2 | 0.91 | 0.48 | 0.95 | 0.4 | 0.44 | 0.96 |
| | 0.3 | 0.93 | 0.33 | 0.98 | 0.52 | 0.4 | 0.95 |
| | 0.4 | 0.93 | 0.2 | 0.99 | 0.53 | 0.29 | 0.94 |
| | 0.5 | 0.93 | 0.1 | 0.99 | 0.59 | 0.17 | 0.94 |
| | 0.6 | 0.93 | 0.08 | 1.0 | 0.75 | 0.14 | 0.94 |
| | 0.7 | 0.93 | 0.03 | 1.0 | 0.78 | 0.06 | 0.93 |
| | 0.8 | 0.93 | 0.01 | 1.0 | 1.0 | 0.03 | 0.93 |
| | 0.9 | 0.93 | 0.0 | 1.0 | 0.0 | 0.0 | 0.93 |
| ICU+ | 0.1 | 0.72 | 0.9 | 0.68 | 0.42 | 0.57 | 0.96 |
| | 0.2 | 0.78 | 0.66 | 0.82 | 0.48 | 0.55 | 0.9 |
| | 0.3 | 0.82 | 0.5 | 0.9 | 0.56 | 0.53 | 0.87 |
| | 0.4 | 0.82 | 0.34 | 0.94 | 0.6 | 0.44 | 0.85 |
| | 0.5 | 0.82 | 0.19 | 0.98 | 0.75 | 0.31 | 0.82 |
| | 0.6 | 0.82 | 0.17 | 0.99 | 0.82 | 0.28 | 0.82 |
| | 0.7 | 0.82 | 0.1 | 1.0 | 1.0 | 0.18 | 0.81 |
| | 0.8 | 0.8 | 0.05 | 1.0 | 1.0 | 0.09 | 0.8 |
| | 0.9 | 0.79 | 0.0 | 1.0 | 0.0 | 0.0 | 0.79 |
| ICU− | 0.1 | 0.88 | 0.66 | 0.89 | 0.26 | 0.37 | 0.98 |
| | 0.2 | 0.93 | 0.42 | 0.96 | 0.37 | 0.39 | 0.97 |
| | 0.3 | 0.95 | 0.29 | 0.98 | 0.52 | 0.38 | 0.96 |
| | 0.4 | 0.95 | 0.15 | 0.99 | 0.49 | 0.23 | 0.95 |
| | 0.5 | 0.95 | 0.08 | 1.0 | 0.52 | 0.14 | 0.95 |
| | 0.6 | 0.95 | 0.05 | 1.0 | 0.67 | 0.09 | 0.95 |
| | 0.7 | 0.95 | 0.01 | 1.0 | 0.4 | 0.02 | 0.95 |
| | 0.8 | 0.95 | 0.0 | 1.0 | 0.0 | 0.0 | 0.95 |
| | 0.9 | 0.95 | 0.0 | 1.0 | 0.0 | 0.0 | 0.95 |
| White | 0.1 | 0.85 | 0.75 | 0.86 | 0.32 | 0.45 | 0.98 |
| | 0.2 | 0.9 | 0.51 | 0.94 | 0.42 | 0.46 | 0.96 |
| | 0.3 | 0.92 | 0.36 | 0.97 | 0.54 | 0.43 | 0.95 |
| | 0.4 | 0.92 | 0.21 | 0.98 | 0.51 | 0.29 | 0.93 |
| | 0.5 | 0.92 | 0.11 | 0.99 | 0.57 | 0.19 | 0.93 |
| | 0.6 | 0.92 | 0.08 | 1.0 | 0.73 | 0.14 | 0.93 |
| | 0.7 | 0.92 | 0.03 | 1.0 | 0.78 | 0.07 | 0.92 |
| | 0.8 | 0.92 | 0.01 | 1.0 | 1.0 | 0.03 | 0.92 |
| | 0.9 | 0.92 | 0.0 | 1.0 | 0.0 | 0.0 | 0.92 |
| Non-White | 0.1 | 0.86 | 0.76 | 0.87 | 0.29 | 0.42 | 0.98 |
| | 0.2 | 0.92 | 0.54 | 0.95 | 0.41 | 0.47 | 0.97 |
| | 0.3 | 0.94 | 0.42 | 0.98 | 0.55 | 0.47 | 0.96 |
| | 0.4 | 0.94 | 0.28 | 0.99 | 0.66 | 0.4 | 0.95 |
| | 0.5 | 0.94 | 0.16 | 1.0 | 0.85 | 0.28 | 0.94 |
| | 0.6 | 0.94 | 0.15 | 1.0 | 0.83 | 0.25 | 0.94 |
| | 0.7 | 0.94 | 0.09 | 1.0 | 0.86 | 0.16 | 0.94 |
| | 0.8 | 0.94 | 0.03 | 1.0 | 1.0 | 0.06 | 0.93 |
| | 0.9 | 0.93 | 0.0 | 1.0 | 0.0 | 0.0 | 0.93 |

Table 3 shows prediction performance under different cut-offs for each subgroup of patients.

In exemplary embodiment, the deep learning algorithm and model shown in FIG. 1 uses EHR data to predict in-hospital mortality, 30-day mortality, or discharge to hospice. The model incorporates both historical and ongoing features in the patient's medical record to generate a numerical risk score on the second day of admission. This machine learning approach was shown to have excellent predictive value in the overall cohort and across multiple subgroups, including patients with COVID−19 infection. In some embodiments, the model can be used iteratively to reanalyze a patient's conditions and predictions. In some further embodiments, the model can also be supplemented with outpatient data to analyze the individual after they have left the hospital. In some further embodiments, the model can also be used to provide support for caregivers to analyze when to send the patient to the hospital to lower the chances of mortality. In some embodiments, this could include critical information for caregivers about helping support patients through serious illness and optimizing quality of life as well as the ability to create an electronic dashboard with caregiver reported symptom burden to proactively reduce caregiver burnout.

This model and system has several unique features. First, the deep learning algorithm and model can be trained with data collected during a pandemic and still be able to analyze both COVID and non-COVID patients. This robustness can handle where case-fatality rates of COVID−19 fluctuate, as well as handling multiple experimental interventions for COVID−19 have entered and exited clinical practice due to lack of demonstrable efficacy. The care of non-COVID patients during a pandemic would also be significantly impacted due to systemic changes in healthcare delivery in response to the pandemic. The model can be continually retrained to account for new therapies that become increasingly standardized and hospitals look to resume pre-pandemic treatment protocols and procedure schedules.

As shown above, the model supports the changing landscape of COVID−19 and its effects on non-COVID care. As shown in FIGS. 3A and 3B, the model performed well with similar AUROCs between COVID+ and COVID-subgroups. In some embodiments, this finding can suggest that many of the predictive factors of short-term mortality or hospice discharge are likely shared between COVID+ and COVID-patients, independent of the COVID−19 diagnosis.

Second, the model is capable of integrating a diverse array of EHR features into a single comprehensive risk score. An average of over five hundred continuous, categorical, and multi-dimensional variables can be extracted and combined for each patient. Furthermore, feature selection does not require expert knowledge or manual curation. After integrating the model into an EHR pipeline, risk scores can be automatically generated on a system-wide level without requiring user input. This allows for the model to capture the breadth of a patient's acute and chronic issues. The EHR-based machine learning model uses data volume and processing power to allow for a large number of variables to be analyzed for making the comprehensive risk score.

Third, the models support training data sets that include a diverse patient population spread across medical, surgical, and subspecialty floors and ICUs at various hospitals. The broad scope of the study cohort suggests that similarly structured machine learning models could be implemented successfully at other institutions. The model also supports excellent positive predictive value across all studied clinical subgroups at relatively modest cut-offs, supporting its generalizability. Setting a threshold score of 0.3, for example, identified patients at risk of short-term mortality or hospice discharge with greater than 50% positive predictive value and 90% specificity in each subgroup as well as the overall cohort. By using the model to identify high-risk patients, clinical care can then be enhanced through increased clinical attention or advance care planning discussions.

In at least one embodiment, one clinical application of the model is through directed palliative care. As a subspecialty that focuses on quality of life, symptom management, and goals of care, palliative care is uniquely positioned to intervene on patients who are at high risk for poor outcomes. Previous research has shown that palliative care interventions on high-risk patients may increase advance care planning, decrease ICU transfers, and facilitate goal-concordant limitations in care. Recent studies have also highlighted the potential of improving palliative care through machine learning techniques. The model can be used to implement a palliative care intervention by using the hospital-wide screening potential of the model. In another embodiment, the inpatient mortality model can also be applied to clinical applications.

The deep learning model described herein is capable of predicting in-hospital mortality, 30-day mortality, or discharge to hospice among inpatients at a large academic and community-based healthcare system. The single model can accurately predict short-term mortality outcomes of patients with and without COVID-19 infection with excellent predictive value.

Furthermore, combining EHR data and administrative claims data to predict mortality of patients assists in better identifying patients who might benefit from palliative care. In this implementation, the LSTM model is used to adapt to the time-series data when the time steps are of arbitrary size. LSTM models can also be used to handle vanishing gradient problems by retaining information (via memory unit) for an arbitrary time period. The memory unit can determine which information needs to be retained, forgotten, and output to the next computational unit.

In addition, a framework for using a LSTM model for mortality prediction has additional advantages, as it (1) is suitable for multiple data format sources, (2) does not require expert knowledge or hand selection to design features, (3) can dynamically update predicted mortality probabilities based on newly added features of patients, and (4) is a more general framework that can be used in both outpatient and inpatient systems. Understanding the relative performance of these models for identifying high-risk patients may help health care systems and managed care organizations select the optimal model for their organization.

The combination of EHR and claims data provides improved accuracy. The combined EHR data and claims data provided more information for the models to discover and identify more potential patterns, which may contribute to the accurate predictions. The advances in system demonstrate a more accurate, holistic approach to identifying patients at high risk of mortality using all available sources of patient information (claims and EHR) for both inpatients and outpatients. This system can also be used to test the impact of palliative care for patients in different phases of care on quality, spending, and utilization. This could allow value-based care organizations to more accurately measure the added value of palliative care for various populations of patients.

Furthermore, effective use of palliative care is critical to the success of value-based care organizations by improving quality and reducing costs for patients near the end of life, where spending is concentrated. However, one of the key barriers to widespread adoption of palliative care to date has been correctly identifying which patients to target. Use of the models and systems described herein can provide improved accuracy in identifying those patients as well as more quickly.

Since, inpatients usually have significantly more events than outpatients, the addition of outpatient support for the model would expand the accuracy as applied to those patients and enable better prediction of mortality. Since the number of records could have an impact on model prediction results. Specifically, prediction results became less accurate for patients with fewer records. The more information (and events) that are supported by the system, the better the accuracy.

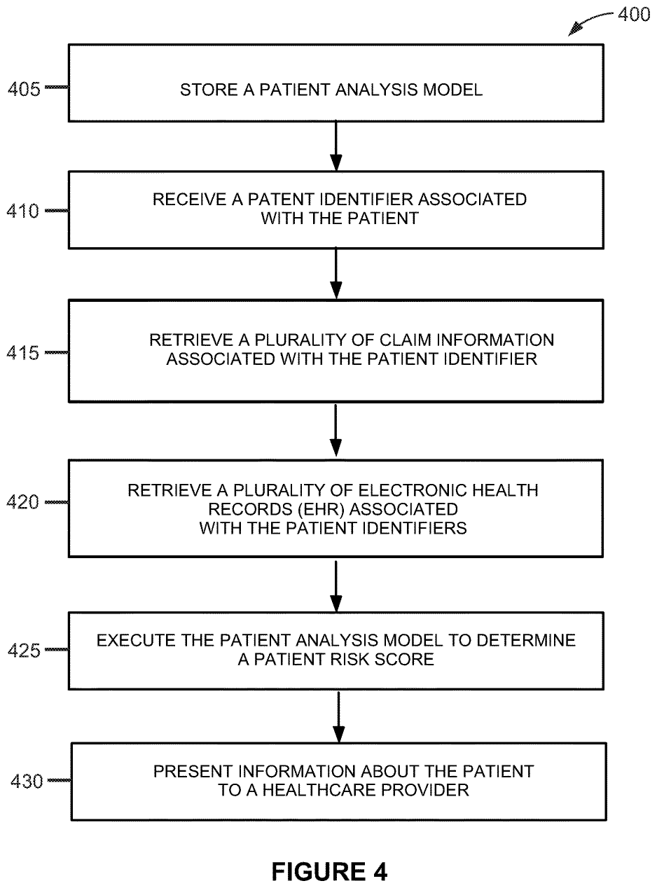

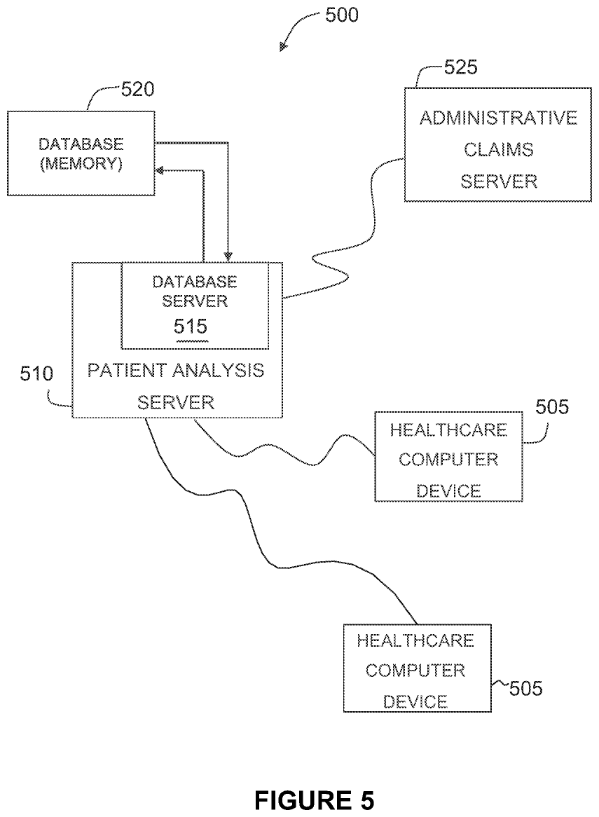

FIG. 4 illustrates a process 400 for analyzing patient health and potential outcomes using the system 500 (shown in FIG. 5). In the exemplary embodiment, process 400 is implemented using the PA server 510 (shown in FIG. 5) in communication with a plurality of healthcare computer devices 505 (shown in FIG. 5) and at least one administrative claims server 525 (shown in FIG. 5).

In the exemplary embodiment, the PA server 510 stores 405 a patient analysis model.

In the exemplary embodiment, the PA server 510 receives 410 a patent identifier associated with the patient, where the patient has been admitted to a hospital.

In the exemplary embodiment, the PA server 510 retrieves 415 a plurality of claim information associated with the patient identifier.

In the exemplary embodiment, the PA server 510 retrieves 420 a plurality of electronic health records (EHR) associated with the patient identifier.

In the exemplary embodiment, the PA server 510 executes 425 the patient analysis model to determine a patient risk score based on the plurality of claim information and the plurality of EHR.

In the exemplary embodiment, PA server 510 presents 430 information about the patient to a healthcare provider, such as via a healthcare computer device 505.

In some embodiments, the PA server 510 determines one or more courses of action for the patient based on the patient risk score and the plurality of her.

The PA server 510 presents the determined one or more courses of action to the healthcare provider, such as via a healthcare computer device 505.

In some further embodiments, the PA server 510 retrieves the plurality of EHR for the patient for the last 24 hours. The PA server 510 then iteratively determines the course of action for the patient.

In some additional embodiments, the course of action includes at least one of palliative care and hospice care.

In some alternative embodiments, the PA server 510 determines a mortality outcome for the patient.

In yet another embodiment, the PA server 510 submits patient information from a current hospital stay to the model.

In still additional embodiments, the PA server 510 receives patient outcome information for the current patient and updates the model with the received patient outcome and the information about the patient.

FIG. 5 depicts a simplified block diagram of an exemplary computer system 500 for implementing process 400 shown in FIG. 4. In the exemplary embodiment, system 500 may be used for predicting patient outcomes. As described below in more detail, a patient analysis (“PA”) server 510 may be configured to store a patient analysis model; receive a patent identifier associated with the patient; retrieve a plurality of claim information associated with the patient identifier; retrieve a plurality of electronic health records (EHR) associated with the patient identifier; execute the patient analysis model to determine a patient risk score based on the plurality of claim information and the plurality of EHR; and present information about the patient to a healthcare provider.

In the exemplary embodiment, healthcare computer devices 505 are computers that include a web browser or a software application, which enables healthcare computer devices 505 to access PA server 510 using the Internet. More specifically, healthcare computer devices 505 are communicatively coupled to the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem. Healthcare computer devices 505 may be any device capable of accessing the Internet including, but not limited to, a mobile device, a desktop computer, a laptop computer, a personal digital assistant (PDA), a cellular phone, a smartphone, a tablet, a phablet, wearable electronics, smart watch, virtual headsets or glasses (e.g., AR (augmented reality), VR (virtual reality), or XR (extended reality) headsets or glasses), chat bots, or other web-based connectable equipment or mobile devices. In at least some embodiments, healthcare computer devices 505 are associated with healthcare providers and are configured to receive and process patient information.

A database server 515 may be communicatively coupled to a database 520 that stores data. In one embodiment, database 520 may include patient information, EHR, models, potential outcomes, and/or user preferences. In the exemplary embodiment, database 520 may be stored remotely from PA server 510. In some embodiments, database 520 may be decentralized. In the exemplary embodiment, a person may access database 520 via stakeholder computer devices 505 or administrative claims server 525 by logging onto IA server 510, as described herein.

PA server 510 may be communicatively coupled with one or more the healthcare computer devices 505. In some embodiments, PA server 510 may be associated with, or is part of a computer network associated with a healthcare system or individual healthcare provider, such as a hospital or health insurance company (not shown). In other embodiments, PA server 510 may be associated with a third party and is merely in communication with the healthcare system or individual healthcare provider. More specifically, PA servers 525 are communicatively coupled to the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem. PA servers 525 may be any device capable of accessing the Internet including, but not limited to, a mobile device, a desktop computer, a laptop computer, a personal digital assistant (PDA), a cellular phone, a smartphone, a tablet, a phablet, wearable electronics, smart watch, virtual headsets or glasses (e.g., AR (augmented reality), VR (virtual reality), or XR (extended reality) headsets or glasses), chat bots, or other web-based connectable equipment or mobile devices.

One or more administrative claims servers 525 may be communicatively coupled with PA server 510 via the Internet or a local network. More specifically, administrative claims servers 525 are communicatively coupled to the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem. Administrative claims servers 525 may be any device capable of accessing the Internet including, but not limited to, a mobile device, a desktop computer, a laptop computer, a personal digital assistant (PDA), a cellular phone, a smartphone, a tablet, a phablet, wearable electronics, smart watch, virtual headsets or glasses (e.g., AR (augmented reality), VR (virtual reality), or XR (extended reality) headsets or glasses), chat bots, or other web-based connectable equipment or mobile devices. In at least one embodiment, administrative claims servers 525 store EHRs and administrative claims for patients.

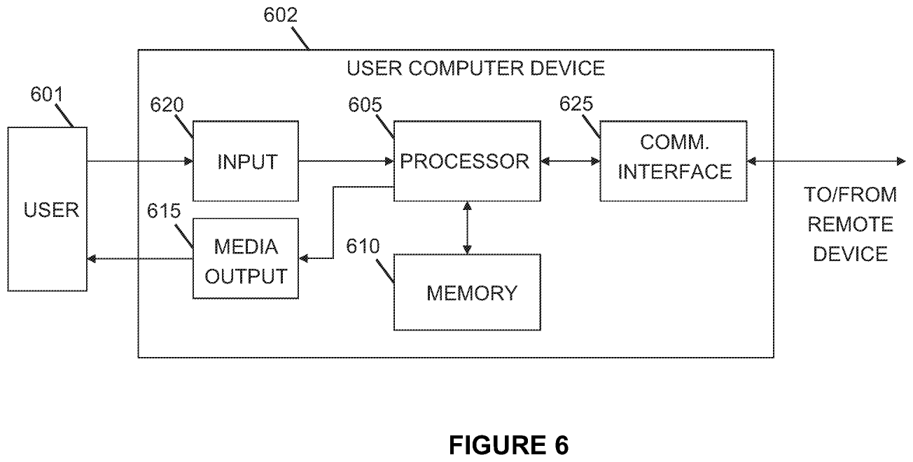

FIG. 6 depicts an exemplary configuration of client computer devices, in accordance with one embodiment of the present disclosure. User computer device 602 may be operated by a user 601. User computer device 602 may include, but is not limited to, healthcare computer device 505 (shown in FIG. 5).

User computer device 602 may include a processor 605 for executing instructions. In some embodiments, executable instructions are stored in a memory area 610. Processor 605 may include one or more processing units (e.g., in a multi-core configuration). Memory area 610 may be any device allowing information such as executable instructions and/or transaction data to be stored and retrieved. Memory area 610 may include one or more computer readable media.

User computer device 602 may also include at least one media output component 615 for presenting information to user 601. Media output component 615 may be any component capable of conveying information to user 601. In some embodiments, media output component 615 may include an output adapter (not shown) such as a video adapter and/or an audio adapter. An output adapter may be operatively coupled to processor 605 and operatively coupleable to an output device such as a display device (e.g., a cathode ray tube (CRT), liquid crystal display (LCD), light emitting diode (LED) display, or “electronic ink” display) or an audio output device (e.g., a speaker or headphones).

In some embodiments, media output component 615 may be configured to present a graphical user interface (e.g., a web browser and/or a client application) to user 601. A graphical user interface may include, for example, potential patient outcomes. In some embodiments, user computer device 602 may include an input device 620 for receiving input from user 601. User 601 may use input device 620 to, without limitation, select and/or enter one or more patient variables.

Input device 620 may include, for example, a keyboard, a pointing device, a mouse, a stylus, a touch sensitive panel (e.g., a touch pad or a touch screen), a gyroscope, an accelerometer, a position detector, a biometric input device, and/or an audio input device. A single component such as a touch screen may function as both an output device of media output component 615 and input device 620.

User computer device 602 may also include a communication interface 625, communicatively coupled to a remote device such as PA server 510 (shown in FIG. 5). Communication interface 625 may include, for example, a wired or wireless network adapter and/or a wireless data transceiver for use with a mobile telecommunications network.

Stored in memory area 610 are, for example, computer readable instructions for providing a user interface to user 601 via media output component 615 and, optionally, receiving and processing input from input device 620. A user interface may include, among other possibilities, a web browser and/or a client application. Web browsers enable users, such as user 601, to display and interact with media and other information typically embedded on a web page or a website from PA server 510. A client application allows user 1101 to interact with, for example, patient variables and patient outcomes. For example, instructions may be stored by a cloud service, and the output of the execution of the instructions sent to the media output component 615.

Processor 605 executes computer-executable instructions for implementing aspects of the disclosure. In some embodiments, the processor 605 is transformed into a special purpose microprocessor by executing computer-executable instructions or by otherwise being programmed. For example, the processor 605 may be programmed with the instructions such as process 400 (shown in FIG. 4).

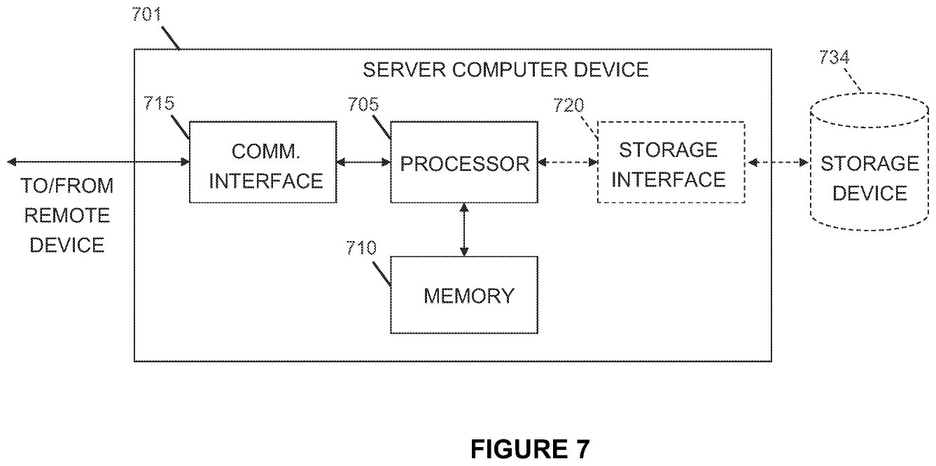

FIG. 7 illustrates an example configuration of the server system, in accordance with one embodiment of the present disclosure. Server computer device 701 may include, but is not limited to, PA server 510, database server 515, and administrative claims server 525 (all shown in FIG. 5). Server computer device 701 also includes a processor 705 for executing instructions. Instructions may be stored in a memory area 710. Processor 705 may include one or more processing units (e.g., in a multi-core configuration).

Processor 705 is operatively coupled to a communication interface 715 such that server computer device 701 is capable of communicating with a remote device such as another server computer device 701, stakeholder computer device 1005 (shown in FIG. 10), or standard organization server 1025. For example, communication interface 715 may receive requests from stakeholder computer devices 1005 via the Internet.

Processor 705 may also be operatively coupled to a storage device 734. Storage device 734 is any computer-operated hardware suitable for storing and/or retrieving data, such as, but not limited to, data associated with database 520 (shown in FIG. 5). In some embodiments, storage device 734 is integrated in server computer device 701. For example, server computer device 701 may include one or more hard disk drives as storage device 734. In other embodiments, storage device 734 is external to server computer device 701 and may be accessed by a plurality of server computer devices 701. For example, storage device 734 may include a storage area network (SAN), a network attached storage (NAS) system, and/or multiple storage units such as hard disks and/or solid state disks in a redundant array of inexpensive disks (RAID) configuration.

In some embodiments, processor 705 is operatively coupled to storage device 734 via a storage interface 720. Storage interface 720 is any component capable of providing processor 705 with access to storage device 734. Storage interface 720 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 705 with access to storage device 734.

Processor 705 executes computer-executable instructions for implementing aspects of the disclosure. In some embodiments, the processor 705 is transformed into a special purpose microprocessor by executing computer-executable instructions or by otherwise being programmed. For example, the processor 705 is programmed with the instructions such as process 400 (shown in FIG. 4)

### Machine Learning & Other Matters

The computer-implemented methods discussed herein may include additional, less, or alternate actions, including those discussed elsewhere herein. The methods may be implemented via one or more local or remote processors, transceivers, servers, and/or sensors, and/or via computer-executable instructions stored on non-transitory computer-readable media or medium.

Additionally, the computer systems discussed herein may include additional, less, or alternate functionality, including that discussed elsewhere herein. The computer systems discussed herein may include or be implemented via computer-executable instructions stored on non-transitory computer-readable media or medium.

In some embodiments, the design system is configured to implement machine learning, such that the neural network “learns” to analyze, organize, and/or process data without being explicitly programmed. Machine learning may be implemented through machine learning (ML) methods and algorithms. In an exemplary embodiment, a machine learning (ML) module is configured to implement ML methods and algorithms. In some embodiments, ML methods and algorithms are applied to data inputs and generate machine learning (ML) outputs. Data inputs may include but are not limited to: analog and digital signals (e.g. sound, light, motion, natural phenomena, etc.) Data inputs may further include: sensor data, image data, video data, EHR data, and claim data. ML outputs may include but are not limited to: digital signals (e.g. information data converted from natural phenomena). ML outputs may further include: speech recognition, image or video recognition, medical diagnoses, statistical or financial models, autonomous vehicle decision-making models, robotics behavior modeling, fraud detection analysis, user input recommendations and personalization, game AI, skill acquisition, targeted marketing, big data visualization, weather forecasting, health care predictions and guidance, and/or information extracted about a computer device, a user, a home, a vehicle, or a party of a transaction. In some embodiments, data inputs may include certain ML outputs.

In some embodiments, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, recurrent neural networks, Monte Carlo search trees, generative adversarial networks, dimensionality reduction, and support vector machines. In various embodiments, the implemented ML methods and algorithms are directed toward at least one of a plurality of categorizations of machine learning, such as supervised learning, unsupervised learning, and reinforcement learning.

In one embodiment, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function which maps outputs to inputs and utilize the predictive function to generate ML outputs based on data inputs. The example inputs and example outputs of the training data may include any of the data inputs or ML outputs described above. For example, a ML module may receive training data comprising data associated with different patients and their corresponding outcomes, generate a model which maps the patient data to the outcome data, and recognize potential future outcomes for patients.

In another embodiment, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship. In an exemplary embodiment, a ML module coupled to or in communication with the design system or integrated as a component of the design system receives unlabeled data comprising event data, financial data, social data, geographic data, cultural data, EHR data, and political data, and the ML module employs an unsupervised learning method such as “clustering” to identify patterns and organize the unlabeled data into meaningful groups. The newly organized data may be used, for example, to extract further information about the potential classifications.

In yet another embodiment, ML methods and algorithms are directed toward reinforcement learning, which involves optimizing outputs based on feedback from a reward signal. Specifically ML methods and algorithms directed toward reinforcement learning may receive a user-defined reward signal definition, receive a data input, utilize a decision-making model to generate a ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In an exemplary embodiment, a ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict optimal constraints.

The computer-implemented methods discussed herein may include additional, less, or alternate actions, including those discussed elsewhere herein. The methods may be implemented via one or more local or remote processors, transceivers, servers, and/or sensors (such as processors, transceivers, servers, and/or sensors mounted on vehicles or mobile devices, or associated with smart infrastructure or remote servers), and/or via computer-executable instructions stored on non-transitory computer-readable media or medium. Additionally, the computer systems discussed herein may include additional, less, or alternate functionality, including that discussed elsewhere herein. The computer systems discussed herein may include or be implemented via computer-executable instructions stored on non-transitory computer-readable media or medium.

As used herein, the term “non-transitory computer-readable media” is intended to be representative of any tangible computer-based device implemented in any method or technology for short-term and long-term storage of information, such as, computer-readable instructions, data structures, program modules and sub-modules, or other data in any device. Therefore, the methods described herein may be encoded as executable instructions embodied in a tangible, non-transitory, computer readable medium, including, without limitation, a storage device and/or a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

The patent claims at the end of this document are not intended to be construed under 35 U.S.C. § 112 (f) unless traditional means-plus-function language is expressly recited, such as “means for” or “step for” language being expressly recited in the claim(s).

This written description uses examples to disclose various implementations, including the best mode, and also to enable any person skilled in the art to practice the various implementations, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system comprising:
a memory device;
a processor in communication with the memory device, the processor configured to:
access patient data from historical electronic health record data, the patient data corresponding to a group of patients with identifiable mortality outcomes within a threshold period following discharge from hospitalization;
extract a plurality of patient features from the patient data;
map the patient data into mapped patient data, the mapped patient data including at least one feature vector for each patient of the group of patients;
divide the mapped patient data into a training data set and a validation data set;
train a plurality of deep-learning patient analysis models with the training data set, the plurality of deep-learning patient analysis models to output a predicted patient mortality outcome based on an electronic health record of a patient;
select a deep-learning patient analysis model of the deep-learning patient analysis models based on an analysis of the deep-learning patient analysis models with the validation data set; and
store the deep-learning patient analysis model in the memory device.

2. The system of claim 1, wherein the predicted patient mortality outcome includes at least one of (1) a first outcome corresponding to dying in a hospital, (2) a second outcome corresponding to being discharged to hospice, and (3) a third outcome corresponding to dying within the threshold period.

3. The system of claim 1, wherein the patient features include diagnosis codes, procedure codes, and medication codes.

4. The system of claim 1, wherein the patient features include age and gender.

5. The system of claim 1, wherein the patient features include at least one lab result and patient vital signs.

6. The system of claim 1, wherein the patient features are arranged in a time increasing order.

7. The system of claim 1, wherein the processor is configured to:
determining, for each patient of the group of patients, a number of healthcare events in the historical electronic health record data; and
for a first patient with a first quantity of healthcare events greater than a threshold quantity:
determine a subset of the first quantity of healthcare events, the subset less than the threshold quantity, the subset including most recent healthcare events of the first patient; and
generate a first feature vector for the first patient based on the subset.

8. The system of claim 7, wherein the processor is configured to, for a second patient with a second quantity of healthcare events less than the threshold quantity:
generate a third quantity of null healthcare events associated with the second patient, a sum of the third quantity and the second quantity equal to the threshold quantity; and
generate a second feature vector for the second patient based on the second quantity of healthcare events and the null healthcare events.

9. The system of claim 1, wherein the deep-learning patient analysis model includes a model layer including:
a first model trained diagnosis codes, procedure codes, and medication codes in the training data set;
a second model trained on lab records in the training data set;
a third model trained on vital signs records in the training data set; and
a fourth model trained on demographic information and social history in the training data set.

10. The system of claim 9, wherein:
the first model, the second model, and the third model are bidirectional long short-term memory models; and
the fourth model is a neural network model.

11. The system of claim 9, wherein the deep-learning patient analysis model includes a concatenation layer that concatenates outputs of the first model, the second model, the third model, and the fourth model into an output vector.

12. The system of claim 9, herein the deep-learning patient analysis model includes an output layer including a binary cross-entropy loss function.

13. A method comprising:
accessing patient data from historical electronic health record data, the patient data corresponding to a group of patients with identifiable mortality outcomes within a threshold period following discharge from hospitalization;
extracting a plurality of patient features from the patient data;
mapping the patient data into mapped patient data, the mapped patient data including at least one feature vector for each patient of the group of patients;
dividing the mapped patient data into a training data set and a validation data set;
training a plurality of deep-learning patient analysis models with the training data set, the plurality of deep-learning patient analysis models to output a predicted patient mortality outcome based on an electronic health record of a patient;
selecting a deep-learning patient analysis model of the deep-learning patient analysis models based on an analysis of the deep-learning patient analysis models with the validation data set; and
storing the deep-learning patient analysis model in a memory device.

14. The method of claim 13, wherein the predicted patient mortality outcome includes at least one of (1) a first outcome corresponding to dying in a hospital, (2) a second outcome corresponding to being discharged to hospice, and (3) a third outcome corresponding to dying within the threshold period.

15. The method of claim 13, further including:
determining, for each patient of the group of patients, a number of healthcare events in the historical electronic health record data; and
for a first patient with a first quantity of healthcare events greater than a threshold quantity:
determining a subset of the first quantity of healthcare events, the subset less than the threshold quantity, the subset including most recent healthcare events of the first patient; and
generating a first feature vector for the first patient based on the subset.

16. The method of claim 13, further including, for a second patient with a second quantity of healthcare events less than the threshold quantity:
generating a third quantity of null healthcare events associated with the second patient, a sum of the third quantity and the second quantity equal to the threshold quantity; and
generating a second feature vector for the second patient based on the second quantity of healthcare events and the null healthcare events.

17. A non-transitory computer readable medium comprising instructions, which when executed, cause a processor to at least:
access patient data from historical electronic health record data, the patient data corresponding to a group of patients with identifiable mortality outcomes within a threshold period following discharge from hospitalization;
extract a plurality of patient features from the patient data;
map the patient data into mapped patient data, the mapped patient data including at least one feature vector for each patient of the group of patients;
divide the mapped patient data into a training data set and a validation data set;
train a plurality of deep-learning patient analysis models with the training data set, the plurality of deep-learning patient analysis models to output a predicted patient mortality outcome based on an electronic health record of a patient;
select a deep-learning patient analysis model of the deep-learning patient analysis models based on an analysis of the deep-learning patient analysis models with the validation data set; and
store the deep-learning patient analysis model in a memory device.

18. The non-transitory computer readable medium of claim 17, wherein the predicted patient mortality outcome includes at least one of (1) a first outcome corresponding to dying in a hospital, (2) a second outcome corresponding to being discharged to hospice, and (3) a third outcome corresponding to dying within the threshold period.

19. The non-transitory computer readable medium of claim 17, wherein the instructions, when executed, cause the processor:
determine, for each patient of the group of patients, a number of healthcare events in the historical electronic health record data; and
for a first patient with a first quantity of healthcare events greater than a threshold quantity:
determine a subset of the first quantity of healthcare events, the subset less than the threshold quantity, the subset including most recent healthcare events of the first patient; and
generate a first feature vector for the first patient based on the subset.

20. The non-transitory computer readable medium of claim 19 wherein the instructions, when executed, cause the processor to, for a second patient with a second quantity of healthcare events less than the threshold quantity:
generate a third quantity of null healthcare events associated with the second patient, a sum of the third quantity and the second quantity equal to the threshold quantity; and
generate a second feature vector for the second patient based on the second quantity of healthcare events and the null healthcare events.
